(19) United States
(12) Patent Application Publication
(10) Pub. No.: US 2003/0108938 A1
(43) Pub. Date: Jun. 12, 2003

(54) Pharmacogenomics-based clinical trial design recommendation and management system and method

(76) Inventors: David Pickar, Chevy Chase, MD (US); Jack D. Hidary, New York, NY (US); Elizabeth Gray, Cabin John, MD (US)

Correspondence Address: MINTZ LEVIN COHN FERRIS GLOVSKY AND POPEO PC, 12010 SUNSET HILLS ROAD, SUITE 900, RESTON, VA, 20190, US

(21) Appl. No.: 10/288,336

(22) Filed: Nov. 6, 2002

Related U.S. Application Data
(60) Provisional application No. 60/338,541, filed on Nov. 6, 2001.
(60) Provisional application No. 60/334,248, filed on Nov. 28, 2001.

(51) Int. Cl. G06F 17/60; C12Q 1/68; G06F 19/00; G01N 33/48; G01N 33/50

(52) U.S. Cl. 435/6; 702/20; 705/3

(57) ABSTRACT

The present invention relates to computer systems and methods for clinical trials for linking biological information including genomic and proteomic information to the conduct and success of the clinical trial process for therapeutic agents. In particular, the present invention relates to computer systems and methods of analyzing genotypes, clinical phenotypes, and clinical trial requirements for providing recommendations for conduct of various phases of clinical trial process. The system may include a genotype database, a clinical database, clinical trial requirements database, an analytical computer, a recommended trial database, a blood bank, and sequencing machines.

### CROSS REFERENCE TO RELATED APPLICATIONS

This application claims priority from U.S. provisional patent application serial No. 60/338,541, filed on Nov. 6, 2001, and Ser. No. 60/334,248, filed on Nov. 28, 2001, each of which is incorporated by reference in its entirety.

### FIELD OF THE INVENTION

The present invention relates to computer implemented systems and methods for facilitating pharmacogenomics-based clinical trial design recommendation and management.

### BACKGROUND OF THE INVENTION

Pharmacogenomics includes identifying gene variants that influence clinical responses to drug and other treatments. Concepts of using pharmacogenomics in clinical trials are generally known (see e.g., U.S. Patent Publication No. 2001/0034023 A1 to Stanton J R, et al., which is incorporated herein by reference in its entirety). This growing area of medicine enables more individualized, science-based treatment decisions. Other aspects of pharmacogenomics include predicting drug response (efficacy) and limiting side effect profiles. The ability to better predict drug response would allow individualized pharmacotherapy that could increase the chance of selecting an optimal drug for each patient and could offer savings in both time and cost of care, and substantially improve a patient's long-term prognosis.

The pharmacogenomic process includes understanding the mechanisms of action of the drug in question, identifying candidate genes based on their involvement in the mechanism of action for the drug or illness risk factor, identifying gene variants, and determining the association of gene variants with findings from clinical trials. A drawback of existing systems for use in clinical trials is the lack of bioinformatics tools that enables efficient use of pharmacogenomics in clinical trials. Another drawback is that the existing systems lack methodologies that provide for establishing individual patient genotypes, including genome wide candidate gene and single nucleotide polymorphisms (SNP's) and detailed clinical information in a unified database to enable the clinical trial development process.

Pharmacogenomics is particularly useful in unraveling genetic bases of “complex” disorders (e.g. hypertension, diabetes, most psychiatric disorders and many cancers) as well as infectious diseases (e.g. AIDS). Complex disorders are diseases without a simple genetic inheritance, but rather those in which genetic factors effect risk phenotype (clinical manifestation), including severity and outcome, and response to pharmacotherapy. The utilization of genetic information in association with the clinical trial process would enable genetically homogenous and targeted clinical trial populations, thereby improving the “signal to noise” ratio. The value of targeted patient populations, selected by genotype of candidate genes derived from a known genomic drug mechanism pathway analysis will enhance efficiency and success rate and enable cost saving. Another drawback in the existing systems is that they lack a bioinformatics system for clinical applications that utilizes genetically selected or targeted patient populations for establishing a pharmacogenomic foundation.

The drug discovery process involves screening large number of compounds for identification of therapeutic targets. It is estimated that 2 of 5,000 compounds identified from the drug discovery process eventually reach the clinical market. Once a lead drug candidate is chosen for clinical development, the clinical trial process involves FDA oversight for Phases I-III. Phase I studies involve short term drug administration to normal volunteers with the goal of establishing pharmacokinetic, preliminary safety and dose finding. Phase II, often performed in two stages, involves the administration of the compound to patients having the medical indication with the goal of establishing preliminary efficacy, safety analysis over longer term administration and dose finding. Phase III involves extensive controlled clinical trial databases which are used as pivotal studies to support the FDA process. Clinicians are often faced with issues of making decisions during all the phases of the clinical study because of the reasons that the clinical study needs to satisfy the requirements set forth by the FDA. However, the existing systems lack bioinformatics features for pharmacogenomics that can examine all Phases of the Drug Development Life Cycle and provide solutions or recommendations to clinicians.

Other drawbacks also exist.

### SUMMARY OF THE INVENTION

The invention overcomes these and other drawbacks in existing systems. One aspect of the invention relates to a bioinformatics system that facilitates use of pharmacogenomics in clinical trials. Another aspect of the invention relates to linking biological information, including genomic and proteomic information, to the conduct and success of the clinical trial process for therapeutic agents.

In one embodiment, the present invention provides an effective system to aid in protocol design, operation, and recommendations for Phase I-III clinical trials which incorporate pharmacogenomic principles and methods.

In another embodiment, the invention provides the system and software to enable a user to select the category of drug to be tested (e.g., antidepressant, anti-hypertensive agents), the specific mechanism of the drug in question within the drug category (e.g., serotonin reuptake inhibitor antidepressant; ACE inhibitor antihypertensive), to receive, in an organized format, and genetic information (eg. gene variants, SNP's, molecular markers, protein markers) including their allelic frequencies, which are related to the mechanism of action and/or have been reported to be associated with outcome measures of the drug under investigation.

In yet another embodiment, the invention further provides for on going patient selection balance; this involves maintaining balanced treatment “arms”, involving patients with specific genotypes, wherein the system ensures sufficient statistical power needed for hypothesis testing.

In a further embodiment, the invention provides for an individual patient's clinical outcome (based on data from the clinical trial) to be merged with a personal genetic database. This combined data approach is essential for pharmacogenomic analysis of an a priori genetic hypothesis.

In an additional embodiment, the invention provides information regarding a pool of patients (identified anonymously) including detailed clinical information relating to their disease state. These patients are also genotyped for variants of candidate genes relevant to their illness or class of drug treatment for which they are candidates. In a parallel embodiment, the invention includes whole genome-wide SNP data. In this fashion, the user of the system of the invention can effectively select patients for prospective pharmacogenetic and clinical studies.

In a further embodiment, the invention is directed to a system for controlling and utilizing genetic variants in pharmacogenetic clinical trials. The system may include a genotype database, a clinical database, an analytical computer, a clinical trial requirements database, filtering and optimization methods for clinical trial recommendation and a recommended trial database.

One aspect of the invention is directed to systems and methods of utilizing genetic variants in pharmacogenetic clinical trials by analyzing a genotype database for appropriate factors. Another aspect of the invention is directed to methods of selecting individual patients for a clinical trial by analyzing the genotypes of the patients in relation to clinical data to identify appropriate candidates. One embodiment associates a selected genotype with a clinical phenotype. Another embodiment filters genotypic and clinical phenotypic inputs based on clinical trial requirements and performs optimization of clinical trial parameters for trial recommendation.

Other objects and features of the present invention will become apparent from the following detailed description considered in connection with the accompanying drawings that disclose embodiments of the present invention. It should be understood, however, that the drawings are designed for purposes of illustration only and not as a definition of the limits of the invention.

### BRIEF DESCRIPTION OF THE FIGURES

FIG. 1 illustrates a pharmacogenomics-based clinical trial recommendation process according to one embodiment of the invention.

FIG. 2A illustrates a system architecture for a pharmacogenomics-based clinical trial recommendation according to one embodiment of the invention.

FIG. 2B illustrates system modules for pharmacogenomics-based clinical trial system according to one embodiment of the invention.

FIG. 2C illustrates system modules and clinical trial requirements for pharmacogenomics-based clinical trial system according to one embodiment of the invention.

FIG. 3A illustrates a process of analysis for clinical trial recommendation based on genotypic and clinical trait input according to one embodiment of the invention.

FIG. 3B illustrates a process of obtaining a clinical trial design and executing a clinical trial according to one embodiment of the invention.

FIG. 4 illustrates integration of pharmacogenomics-based clinical trial recommendation system with integrated healthcare management system according to one embodiment of the invention.

FIG. 5A illustrates an interface for pharmacogenomics-based clinical trial recommendation system according to one embodiment of the invention.

FIG. 5B illustrates an interface for clinical input of pharmacogenomics-based clinical trial recommendation system according to one embodiment of the invention.

FIG. 5C illustrates an interface for genetic input of pharmacogenomics-based clinical trial recommendation system according to one embodiment of the invention.

FIG. 5D illustrates an interface for inputs filtering of pharmacogenomics-based clinical trial recommendation system according to one embodiment of the invention.

FIG. 5E illustrates an interface for optimizing trial parameters of pharmacogenomics-based clinical trial recommendation system according to one embodiment of the invention.

### DETAILED DESCRIPTION OF THE INVENTION

The present invention relates to systems and methods for clinical trials that link biological information, including genomic and proteomic information, to the conduct and success of the clinical trial process for therapeutic agents. In particular, the present invention relates to systems and methods of analyzing genotypes, clinical phenotypes, and clinical trial requirements to provide recommendations for conducting various phases of clinical trial process.

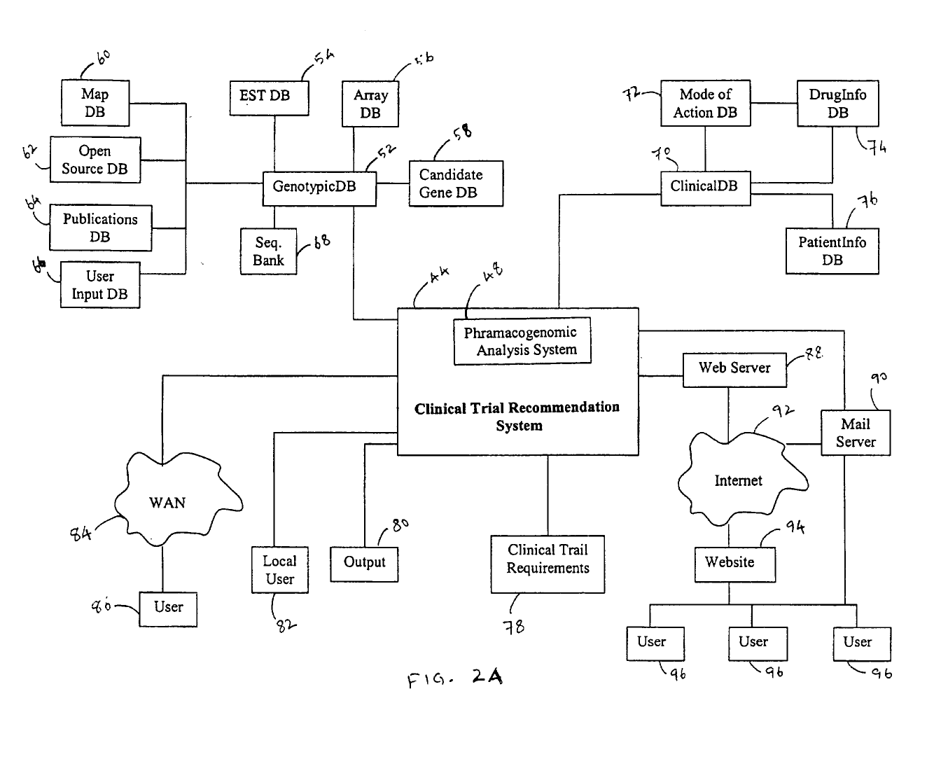

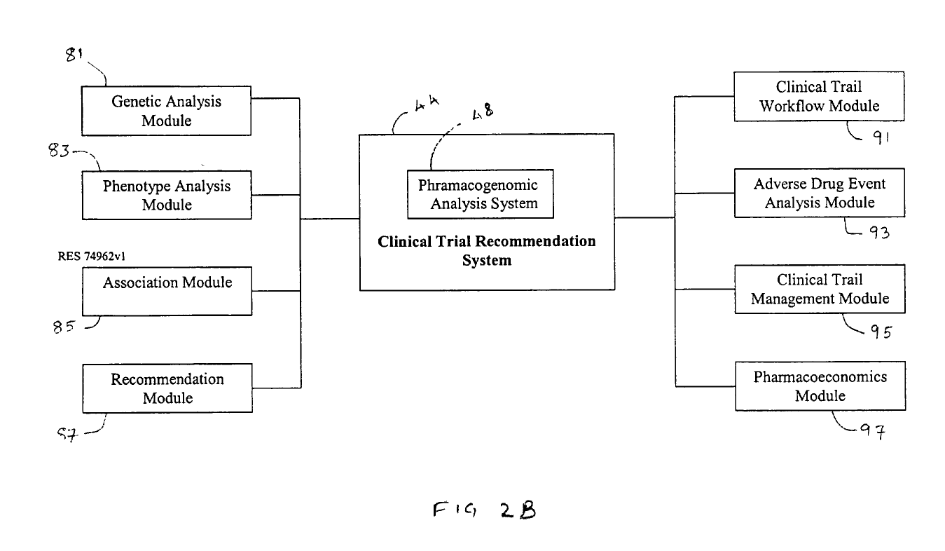

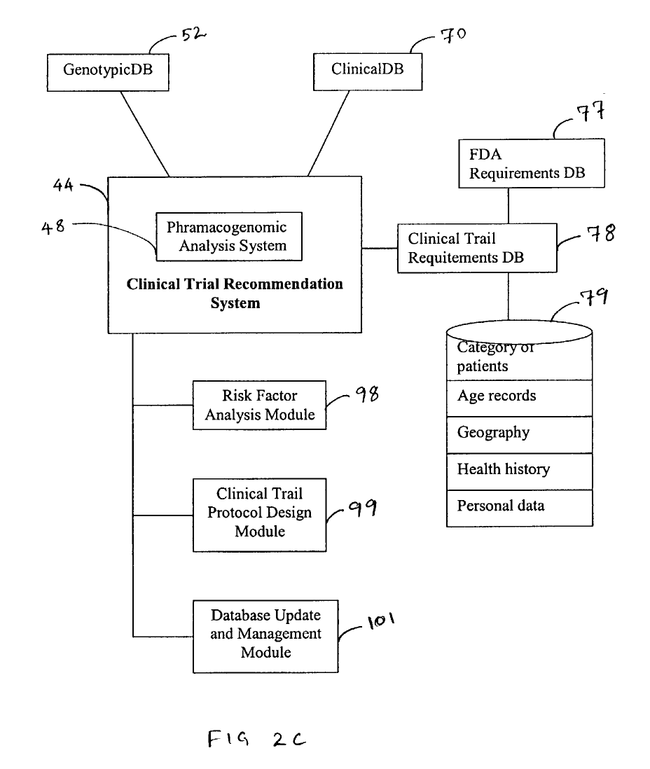

According to one aspect of the invention, as illustrated in FIG. 2A, a clinical trial recommendation (CTR) system 44 may include a pharmacogenomic analysis system 48 that may be used to perform genomic analysis (e.g., associating genotype with phenotype, nucleotide sequence comparison, pattern matching) and proteomic analysis (e.g., protein sequence matching, three dimensional modeling). The CTR system 44 may access and retrieve genotypic data from a genotypic database 52 and clinical data from a clinical database 70.

In one embodiment, the CTR system 44 may permit the utilization of the genotype data to carry out, design and monitor clinical trials. The genotypic database 52 may refer to databases designed to store the genotype data. Such data may include, but are not limited to, groups of individuals patients in whom genotype analysis for common and rare variants, including single nucleotide polymorphisms, have been determined for distinct candidate genes. This data may also include genome-wide SNP maps for individual patients. The genotypic database 52 may include or otherwise access expressed sequence information from an EST (Expressed Sequence Tag) database 54, microarray data from an array database 56, and/or candidate gene data from a candidate gene database 58. The genotypic database 52 may also include or otherwise access genetic sequence (eg. nucleotide sequence, peptide sequence) from sequence bank 68. This sequence bank 68 may be able to store a large volume of genetic data including terra bytes and peta bytes of data. In one embodiment sequence bank 68 may directly access sequence data from genetic sequencing devices. In addtion, the genotypic database 52 may be coupled to other databases including map database 60, open source database 62, publications database 64, and/or user input database 66. Map database 60 may store, for example, information on genetic, physical and transcriptome maps of human and other organisms. Open source databases 62 may include, for example, public databases such as GenBank and SwissProt. The publications 64 database may include, for example, various publications including genomics, proteomics, and clinical trials. User Input database 66 may store any information specified by clinical user. The genotypic database 52 may also be coupled to proprietary databases such as, for example, Celera genomic database (not shown in figure).

The clinical database 70 may include clinical data such as, but not limited to, diagnoses confirmed by standardized assessment tools, confirmed tissue (e.g., tumor) leading to a specific disease diagnosis, illness severity, outcome for illness or syndrome, response to prior drug treatment, family and clinical genetic history, and other elements which contribute to a clinical phenotype and are associated with specific genotypes.

The clinical database 70 may include or otherwise access patient information database 76, mode of action database 72, and/or drug information database 74. Patient information database 76 may include, for example, patient information including medical history, demographical and biographical information (eg. age, sex). The mode of action database 72 may include information regarding drug mechanisms. In some embodiments, the mode of action database 72 may include information on partial understanding of a drug mechanism for example. In other embodiments, the mode of action database 72 may provide drug mechanisms which are speculative for example. The drug information database 74 may, for example, include a list of manufacturers of a drug, dosage information, and results of previous study.

According one embodiment, the pharmacogenomics based CTR system 44 may include a recommended trial database (not shown in Figures). The recommendation trial database may include to an admixture of clinical phenotype and genotypic data such that a patient, or group of patients, may be rapidly selected on the basis of either clinical or genotypic data to serve the needs of a given clinical trial. In this fashion, a unique database may be applied to a distinct clinical trial.

According to another embodiment, the pharmacogenomics based CTR system 44 may access data (e.g., patient blood group, patient DNA source) from a blood bank (not shown in FIG). The blood bank may have a storage facility in which whole blood or other tissues are received from patients who enter the database. This facility may allow, for example, the extraction of DNA of leukocytes, immortalization of cell lines for future DNA extraction or the maintenance of tissue for RNA expression studies.

According to yet another embodiment, the CTR system 44 may be coupled to a plurality of sequencing machines (not shown in figures). The sequencing machines may access biological samples of the blood bank. The sequencing machines may include analytic machines which provide for high throughput genotyping for individual candidate genes, including deep sequencing for rarely occurring single nucleotide polymorphisms or other variants.

According to another aspect of the invention, the pharmacogenomics based clinical trial recommendation CTR system 44 may include a clinical trial requirements database 78. Clinical trial requirements database 78 database may include, for example, one or more inclusion and exclusion criteria for a plurality of clinical protocols. This criteria may include, for example, diagnosis, gender, age, illness severity, prior treatments, etc. In one embodiment, the clinical trial requirements database 78 may include or otherwise access FDA guidelines data.

According to yet another aspect of the invention, the pharmacogenomics based clinical trial recommendation system 44 may be accessed by authorized users of contract research organizations (CROs) who are involved in administering clinical trials.

According to one embodiment of the invention, as illustrated in FIG. 2B, the CTR system 44 may include a plurality of modules for pharmacogenomics based clinical trial system. One or more genetic analysis modules 81 may be able to perform genetic analysis such as, for example, DNA sequence analysis, protein sequence analysis, genetic finger printing analysis, genetic variability analysis, haplotype analysis and phylogenetic analysis. One or more phenotypic analysis modules 83 may be able to perform conventional analysis on phenotypes such as, for example, analysis of drug response, and analysis of disease progression and intensity. One or more association modules 85 may be connected to geneotypic database 52, and clinical database 70 and may be able to determine an association between genetic information in the genotypic database 52 and clinical phenotypic information in the clinical database 70 for a plurality of patients. One or more recommendation modules 87 may be connected to genotypic database 52, clinical phenotypic database 70, and clinical trial requirement database 78 and may be able to provide clinical trial recommendations utilizing the genetic information, the clinical phenotypic information, the clinical trial requirement information and the determined association between the clinical information and the genetic information. The CTR system 44 may be able to store output of clinical trial recommendations.

According to another embodiment of the invention, as illustrated in FIG. 2B, the CTR system 44 may further include one or more clinical workflow modules 91 for monitoring workflow during clinical trial process, one or more adverse drug event modules 93 for analyzing genetic basis of adverse reaction to a plurality of drugs, one or more clinical trial management module 95 for administration of one or more aspects of one or more clinical trial phases (Phases I-IV), and one or more pharmacoeconomics modules 97 for micro- and macro-economic aspects of clinical trials including financing and budgeting.

According to one embodiment of the invention, as illustrated in FIG. 2C, the clinical trial requirements database 78 of the CTR system 44, may include or otherwise access a Food and Drug Administration (FDA) requirements database 77 and a patient database 79. FDA requirements database 77 may include information such as FDA regulations and guidelines for clinical trials. The patient database 79 may include a plurality of data on patients, for example, category of patients, age information, geography, health history, and personal data. The examples of category of patients may include, for example, child, elderly, sex, ethnicity, cognitively impaired individuals, or people who are economically or educationally disadvantaged. In one embodiment, the CTR system 44 may be able to relate data within the patient database 79 using data relation modules (not shown in the figure) for determining an inter-relationship between data. For example, the CTR system 44 may be able to determine child based on age and geography (eg. state). In general, state laws define what constitutes a “child”, and such definitions dictate whether or not a person can legally consent to participate in a clinical trial.

According to another embodiment of the invention, as illustrated in FIG. 2C, the CTR system 44 may also include risk factor analysis module 98, clinical trial protocol design module 99, and database update and management module 101. Risk factor analysis module 98 may be used to predict risks or adverse effects for one or more selected individuals using information from genotypic database 52, clinical database 70, and clinical trial requirement database 78. In one embodiment, the CTR system 44 may be used to predict risks or adverse effects by relating one or more genetically selected individuals for one or more clinical traits with the data in clinical trial requirements database 78. In another embodiment, the CTR system 44 may use a plurality of statistical algorithms for predicting risks or adverse effects. Clinical trial protocol design module 99 may be used to design a protocol for clinical trial. In some embodiments, the clinical trial protocol design module 99 may access FDA requirements database 77 for obtaining FDA guidelines. In other embodiments, the clinical trial protocol design module 99 may access with genotypic database 52, clinical database 70, and clinical trial requirement database 78. In yet other embodiments, the clinical trial protocol design module 99 may utilize the information on risks or adverse effects predicted by the CTR system 44. In one embodiment, database update and management module 101 may periodically update a plurality of databases connected to the CTR system 44 with new data. In another embodiment, the CTR system 44 may maintain the plurality of databases of the invention (e.g., genotypic database, clinical database, clinical trial database) according to a plurality of user enabled set of instructions.

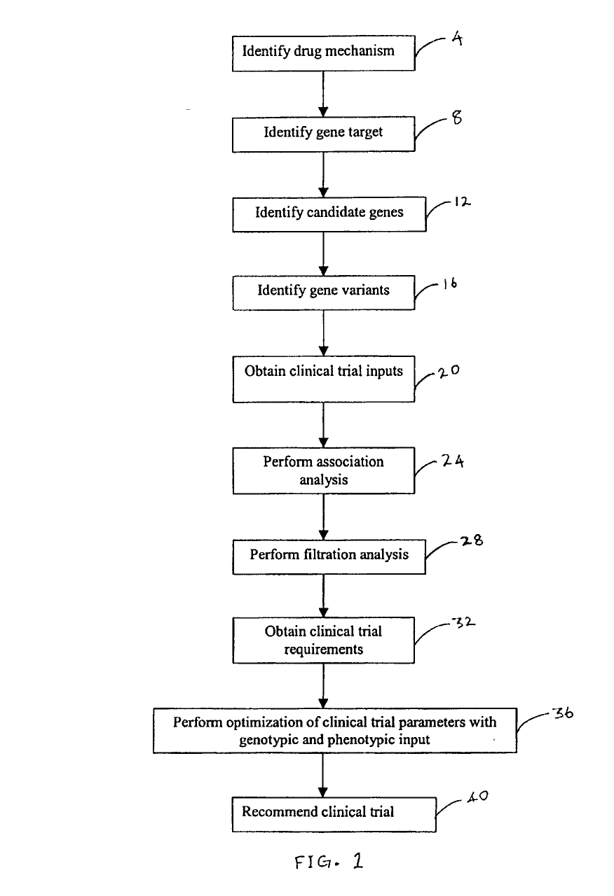

FIG. 1 illustrates a clinical trial recommendation process using pharmacogenomic information. Components of the pharmacogenomics-based clinical trial recommendation process may include drug mechanism analysis, target analysis, candidate gene analysis, gene variant analysis, preliminary clinical trial analysis, association analysis, filtration analysis, clinical trial requirement analysis, and optimization of clinical trial parameters. One advantage of the present invention is that it provides assistance and guidance in managing and maximizing the efficiency of the clinical process using pharmacogenomics.

As illustrated in step 4 of FIG. 1, drug mechanisms may be identified from the mode of action database 72. The drug mechanisms included in the mode of action database 72 may provide insight into the pharmacological processes by which a drug produces its therapeutic effects. Such drug mechanisms include, for example, alterations in function, of components of dopamine systems in the central nervous system in the case of antipsychotic drugs, of cardiac adrenergic systems for some classes of antihypertensive agents, or bacterial genome expression for some antibiotics. In some embodiments, the mode of action database 72 may provide information on partial understanding of a drug mechanism. In other embodiments, the mode of action database 72 may provide drug mechanisms which are speculative.

As shown in step 8 of FIG. 1, gene targets may be identified using the CTR system 44. In one embodiment, gene targets may be included in the genotypic database 52 to provide information regarding a drug's mechanism of action and to provide the basis for pharmacogenetics clinical trials. Such targets include, for example, the D₂ dopamine receptor as a target for antipsychotic compounds or the beta adrenergic receptor for certain antihypertensive agents.

According to one embodiment, candidate genes may be included in the candidate gene database 58 to provide the link between a target (e.g., receptor, enzyme) and its genetic control of target function and production. These candidate genes may be identified from the database in step 12.

According to another embodiment, gene variants may be included in the database to provide the genetic basis for pharmacogenetics studies. For example, the gene that codes for the D₂ receptor exists with common variants (>1% of the population) in the promoter as well as in coding regions. These variants alter an individual's production or composition of the receptor which renders this an excellent target for pharmacogenomic exploration. These gene variants may be identified in step 16 from the genotypic database 52 using the CTR system 44. The gene variants may be due to, but are not limited to SNPs (Single Nucleotide Polymorphisms), variation in candidate genes, variation in number of nucleotide repeats (eg. simple sequence repeats), variation in length of nucleotide repeats, RFLPs (Restriction Fragment Length Polymorphisms), variation in protein sequences and variation in protein structures.

According to yet another embodiment, as shown in step 20, clinical trial inputs may be identified from clinical trial database 70. The clinical trial inputs may include information on one or more clinical phenotypes (e.g., mild cognitive impairment).

According to additional embodiment, an association may be established as shown in step 24 between one or more gene variants and one or more phenotypes. Once the association is determined through association analysis as shown in step 24, a priori hypothesis testing in further clinical trials can be accomplished. According to one embodiment of the invention, the association may be determined using a plurality of statistical methods. In one example, pearson's correlation is used to determined the association between a genotype and clinical phenotype.

According to further embodiment, the CTR system 44 may present associations between genetic information and clinical information and associated genotypes and phenotypes using a plurality of presentation tools in graphical user interface (not shown in FIG. 1). In one embodiment, as shown in step 28, these associations may be filtered using pre-determined statistical significance or threshold values known to one skilled in the art. In another embodiment, the information may be filtered based on genes or phenotypes. For example, a user may be interested in a particular gene selected from several genes showing association for a clinical trait. In this case, the user may be able to select one or more preferred genes and filter out the genes and other information related to the genes which are not preferred.

According one embodiment, the CTR system 44 may be used to obtain a plurality of clinical trial requirements as shown in step 32 . The clinical trial requirements may include, for example, Food and Drug Administration guidelines for various phases of clinical trials. The clinical trial requirements may correspond to, for example, diagnosis, gender, age, illness severity, and/or prior treatments of clinical patients. The CTR system 44 may be used to perform optimization of the plurality of clinical trial requirements using the genotypic and the phenotypic input as shown in step 36. For example, the CTR system 44 may be used to optimize the clinical trial requirements for children at the age group of 10-14 since the clinical trial requirements may be dependent on risk factors in a developmental stage or age of the clinical patients. The CTR system 44 may provide clinical trial recommendation, as shown in step 40, utilizing the results of the optimization.

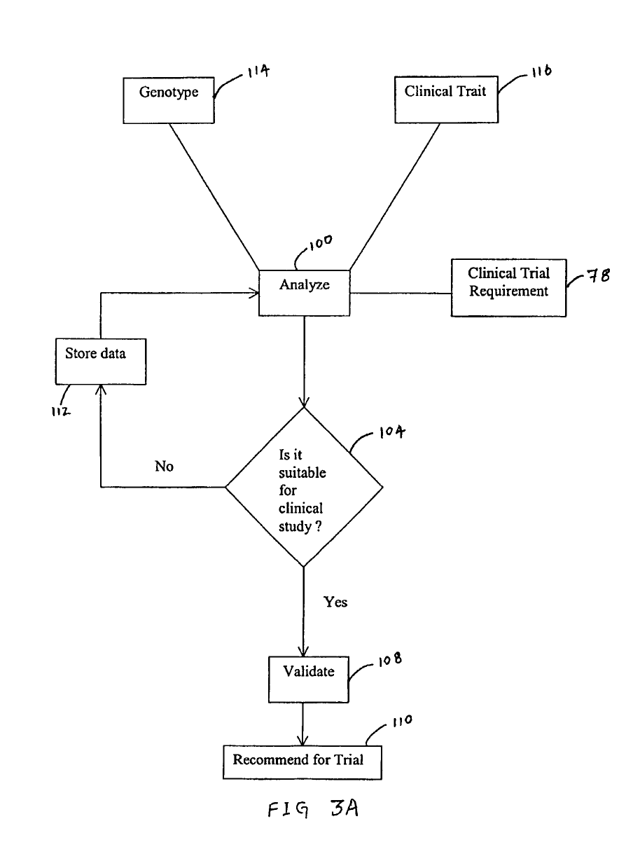

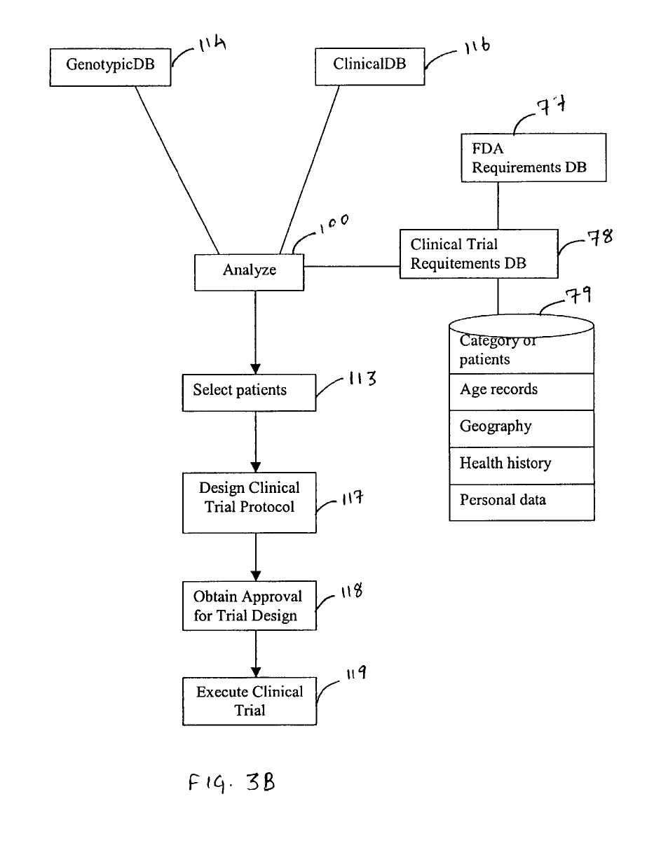

According to one embodiment of the invention, a process for determining a clinical trial recommendation based on genotypic and clinical trait input is illustrated in FIG. 3A. For example, in a clinical study, a plurality of genotypes 114, a plurality of clinical traits 116, and a plurality of clinical trial requirements 78 may be analyzed at step 100 using one or more analytical processors. The clinical trait may include any clinical phenotype such as response to drug, dosage of drug, patient age etc. In this analysis, individuals having similar genotypes and similar clinical traits may be selected and grouped together. For example, one or more selective genotypes may be associated with one or more selective phenotypes. The selected genotypes or clinical traits may be included or excluded depending on the nature of the clinical study. In one embodiment, genotypes with high similarity may be included for a clinical study. In another embodiment, dissimilar genotypes may be included for a clinical study. In yet another embodiment, genotypes may be randomly chosen to have genetic balance, and included in a clinical study. In a further embodiment, the invention provides for ongoing patient selection balance. This may involve maintaining balanced treatment “arms”, involving patients with specific genotypes, thereby ensuring sufficient statistical power needed for hypothesis testing.

The selected genotypes and clinical traits may be analyzed at step 100 with the plurality of clinical trial requirements 78 of a given clinical study. If the selected genotypes and clinical traits meet the clinical trial requirements, they may be validated at step 108 against the plurality of clinical trial requirements of individual phases (e.g., Phase III) of a clinical trial. The trials may be recommended based on output of analysis. If selected genotypes and clinical traits do not meet the clinical trial requirements, the results may be stored at step 112 and may be used for further analysis.

According to another embodiment of the invention, the process of obtaining a clinical trial design and executing a clinical trial are illustrated in FIG. 3B. For example, in a clinical study, a plurality of genotypes 114, a plurality of clinical traits 116, and a plurality of clinical trial requirements 78 may be analyzed at step 100 using one or more analytical processors. In this analysis, individuals having similar genotypes and similar clinical traits may be selected and grouped together as shown in step 113. Clinical trial protocol may be designed for a trial involving selected individuals as shown in step 118. The protocol may consider a plurality of parameters including, for example, risk or adverse drug effect information for selected individual, patient category, sex, age, geography, health history and personal data. The protocol may be submitted electronically to a group of authorized individuals (eg. Institutional Review Board) for review and approval (not shown in FIG. 3B). In some embodiments, a user may authorize a group of individuals to access one or more of the features of the system 44 or one or more of the features connected to the system 44 as part of review and approval of the protocol. After obtaining the approval for the protocol as shown in step 118, the protocol may be executed using the CTR system 44 as shown in step 119.

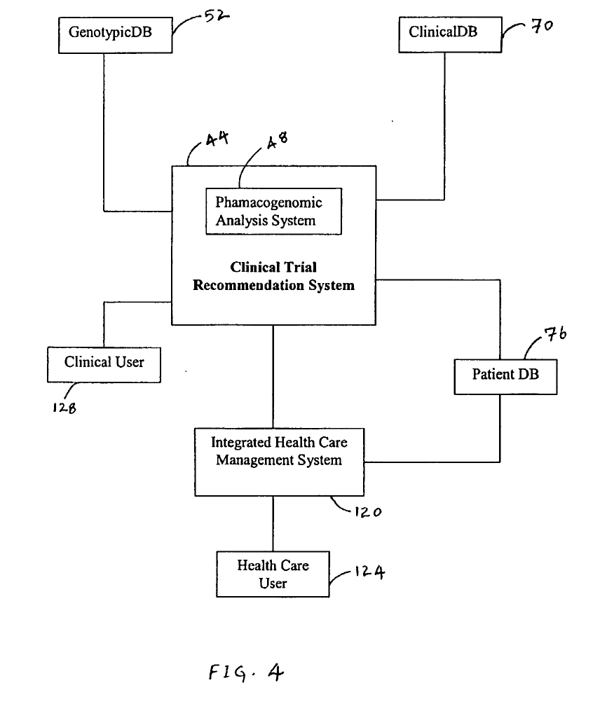

According to another embodiment, as illustrated in FIG. 4, the CTR system 44 may be integrated with an integrated health care management system 120. The integrated healthcare management system 120 may, for example, to a system interact with one or more organizations for managed care systems (eg. PPO, HMO), and a plurality of healthcare users 124 such as healthcare managers, paramedical specialists and physicians. In some embodiments, the healthcare users 124 may have access to a clinical trial recommendation system.

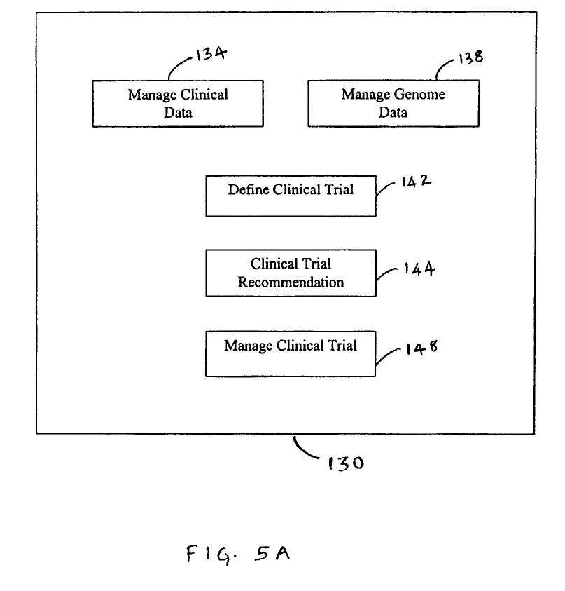

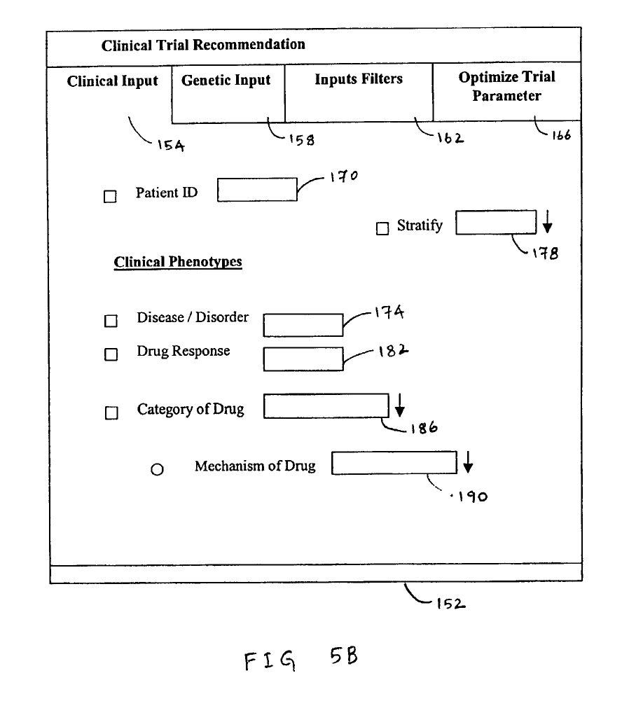

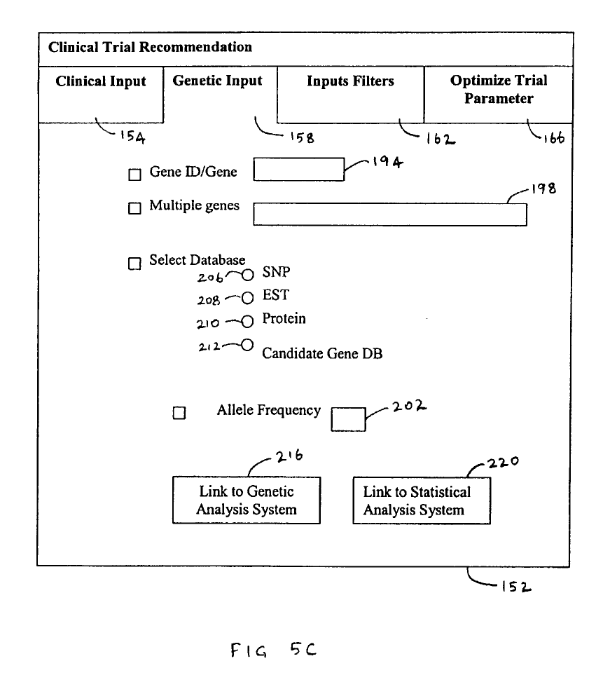

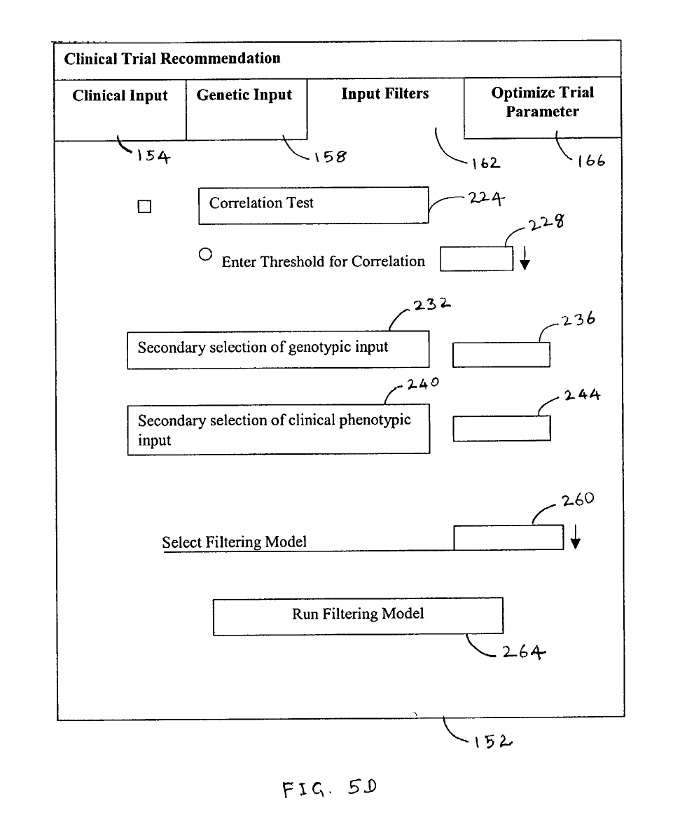

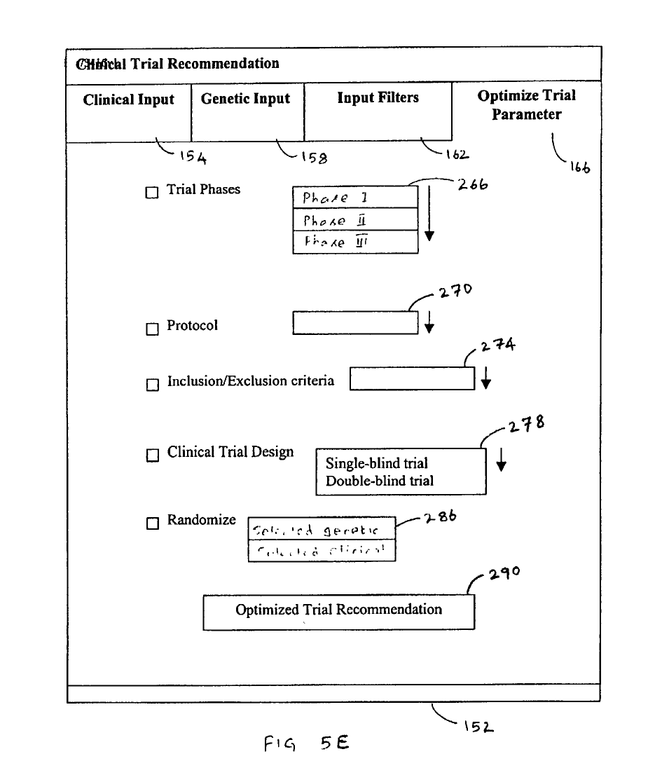

FIG. 5A illustrates a user interface 130 for clinical trial recommendation system of FIG. 4, item 44, according to one embodiment of the invention. The user interface 130 may have a plurality of icons (e.g., clickable buttons) for managing clinical data 134, managing genomic data 138, defining clinical trial 142, recommending clinical trial 144 and managing clinical trial 148. Manage clinical data button 134 may be used to access database management features of pharmaceutical, patient, and other clinical phenotypic databases, for example, in the CTR system 44. Clinical database management features may support entry and editing of data in the clinical databases. The relationships among data and databases may also be managed using these features. In one embodiment, the clinical database management features may include user intervened data update features. In another embodiment, the clinical database may be managed and updated automatically without user intervention. In some embodiments, the clinical database management features may include a plurality of frames preferably in a graphical user interface for performing database maintenance functions.

Manage genome data button 138 may be used to access genetic data (eg. nucleotide sequence, protein sequence, protein structural data, protein functional data, genome map) and publications and reports relevant to genetic data of both proprietary and public databases, for example. Furthermore, the user may operate genome database management features through manage genome data button 138 for entering and editing of data in the genomic or genetic databases of the system 44. For example, the user may manage the relationships among genetic data and databases. In one embodiment, the genome database management features may include user intervened data update features. In another embodiment, the genome database may be managed and updated automatically without user intervention. In some embodiments, the genome database management features may include plurality of frames preferably in graphical user interface for performing database maintenance functions.

A clinical trial may be defined using the define clinical trial button 142. This button 142 may be used to access a plurality of frames, wherein trial information may be recorded and stored. In some embodiments, the system may have a pre-determined format for entering clinical trial information. In other embodiments, the user may be able to create the formats. These formats may correspond to FDA requirements for clinical trials. In one embodiment, the present invention provides an effective system to aid in protocol design, operation, and recommendation for Phase I-III clinical trials and post-market surveillance that utilize pharmacogenomic principles and methods.

Manage clinical trial data button 148 may be coupled to database management features to manage data during the clinical trial. For example, trial status, diagnoses, treatments, and outcomes may be managed. According to one embodiment, clinical trial management features may support data imported from other data systems containing patient data or direct input. A plurality of import/edit screens may be used to show how the clinical trial is being managed.

Clinical trial recommendation button 144, may be used to view an interface for clinical trial recommendation 152 as illustrated in FIGS. 5B, 5C, 5D, and 5E. The clinical trial recommendation interface 152 may have means for inputting, for example, clinical and genetic information, filtering the information and optimizing trial parameters for trial recommendation. For example, the interface 152 of FIGS. 5B, 5C, 5D, and 5E may include user selectable frames such as clinical input 154, genetic input 158, input filters 162 and optimize trial parameters 166 in graphical user interface. According to one embodiment, a plurality of clinical phenotypic records may be obtained, analyzed and managed using clinical input frame 154 as illustrated in FIG. B. The clinical input interface 154 may include a plurality of options for the user to select one or more clinical phenotypic traits or enter a clinical phenotypic trait to be used in the clinical trial. The examples of the clinical phenotypic traits may include, for example, diseases (eg. Alzheimer), disorders (eg. cognitive impairment), drugs (eg. dopamine), categories of drugs (eg. antidepressant, anti-hypertensive agents), mechanisms of drugs (eg. serotonin reuptake inhibitor antidepressant; ACE inhibitor antihypertensive). As illustrated in FIG. 5B, according one embodiment, the user may enter patient ID in box 170 and retrieve individual patient data including patient phenotypic data from patient database 76. In another embodiment, the user may select a clinical phenotypic trait and analyze clinical phenotypic information of group of patients using clinical input frame 154. For example, the user may enter disease phenotype in box 174 and retrieve disease data from the clinical database 70. The disease data may include, but are not limited to, symptoms of disease, diagnostic information and treatment information. Similarly, the user may enter drug response phenotype in box 182 and retrieve drug data from the drug information database 74.

According to another aspect of the invention, the user may input drug related information such as, for example, category of drug, mechanism of drug, etc. In one embodiment, the user may select a drug category from scroll down menu 186. In another embodiment, the user may select a drug mechanism using scroll down menu 190. The system 44 may obtain the information related to selected drug category or drug mechanism from the drug database 74. In addition, the user may stratify the selected clinical phenotypic traits based on a plurality of statistical models known in the art for stratification. The user may use scroll down menu 178 for selecting a statistical model for stratification. In one embodiment, the statistical model for stratification may correspond to phenotypic correlation of individuals. In another embodiment, the statistical model for stratification may correspond to chi-square methodology for grouping individuals. Stratification of individuals based on their clinical phenotypic traits may enable clinicians to target clinical study to a group of individuals with similar clinical phenotype.

According to one embodiment, the user may enter information regarding genetic markers that pertain to biological mechanism of a specific drug undergoing clinical trial and the CTR system may balance distributions of genotypes among study populations undergoing specific clinical trials. Thus, the invention provides the ability to monitor the composition of clinical trial populations during the conduct for the clinical trial.

According to one embodiment, the user may select individual patients who are suitable for a clinical trial on the basis of already performed genotypes. For example, the user may first enter the category of drug in a trial (e.g., antidepressant, anti-epileptic, etc), may next select a specific pathway of its mechanisms (e.g., serotonin reuptake blockage) or describe a pathway not yet existing in the data base, and finally may identify known candidate genes and their variants in the database which could pertain to the drugs therapeutic action on the basis of information.

The genetic input of clinical trial recommendation is illustrated in FIG. 5C. According to one aspect of the present invention, the user may select one or more genetic input from the genetic input frame 158. In one embodiment, the user may enter a gene identification number or a gene name in box 194 and obtain a plurality of information related to the specified gene from the genotypic database 52. In another embodiment, the user may enter more than one gene or multiple genes in box 198 and obtain information related to multiple genes from genotypic database 52. The information on multiple genes may correspond to clinical studies of complex diseases since the complex diseases are known to be controlled by multiple genes. In yet another embodiment, the user may select a plurality of database sources for obtaining genetic data. The genetic data may include, but are not limited to, SNP (single nucleotide polymorphism), EST (Expressed Sequence Tags), protein data, and candidate genes. These data may be obtained from one or more databases such as Seq. Bank 68, EST DB 54, and candidate gene DB 58 of system 44. The genetic input frame 158 may have a link to a genetic analysis system 216, wherein the genetic analysis system 216 enables the user to perform genomic (eg. sequence matching and gene identification, gene expression analysis, genotype analysis) and proteomic (protein identification, predicting protein structure, predicting protein-protein interactions) analysis. The genetic input frame 158 may also have link to a statistical analysis system 220, wherein, the statistical analysis system 220 enables the user to analyze genetic data using plurality of statistical or mathematical methods (eg. principal component method for gene expression, regression methods for genotype association, Hidden-Markov methods for sequence matching). The statistical analysis system may enable the user to group or stratify individuals based on a plurality of genetic similarities. In some embodiments, the selected genes may be allelic variants. The allele frequency selected genes may be displayed in box 202.

According to another aspect of the invention, as illustrated in FIG. 5D, the user may associate the selected genetic inputs with the selected clinical phenotypic inputs. These associations may be determined using one or more of statistical tests. For example, the user may perform correlation test as shown in box 224 of FIG. 5D. The association may be performed between one or more genes including allelic variants and one or more clinical phenotypic traits. The user may filter the associations using a plurality threshold levels for selecting the associated samples. For example, in one embodiment, the threshold level for correlation may be selected from box 228. In some embodiments, the threshold levels may be predetermined. Clinicians and researchers involved in clinical trial may be interested in focusing on a few genes or selecting a few genes. Similarly, they may be interested in a few aspects of information relevant to phenotypic traits. According to one embodiment of the invention as illustrated in FIG. 5D, the user may filter the selected clinical and genetic inputs and the retrieved information related to the selected clinical and genetic inputs. The genetic input may be further selected from box 232. The further selected genetic input may be displayed in box 236. Similarly, the clinical phenotypic input may be further selected from box 240 and the further selected phenotypic input may be displayed in box 244. According to one embodiment of the invention, the user may filter the inputs using one or more filtering models. The filtering models may include parameters such as, for example, a threshold level for association between genetic input and clinical input, a threshold level for determining a similarity between the selected genetic or phenotypic input and the retrieved information from one or more databases in the system 44. According to one aspect of the invention, when the user knows which candidates are pertinent to the drug trial, the CTR system 44 may enable the choice of specific patients, that are already categorized by patterns of candidate gene variants and/or single nucleotide polymorphism (SNP) patterns. In another aspect of the invention, the CTR system 44 may enable the organizers and managers of clinical trials to establish and select pre-hoc trial populations which enable hypotheses of genetic variants as predictors of therapeutic response to be tested in an efficient and scientifically rigorous fashion.

According to another aspect of the invention, the system provides optimization features for clinical trials. As illustrated in FIG. 5E, the optimization trial parameter frame 166 may include a plurality of optimization parameters, wherein the optimization parameters correspond to a plurality of clinical trial requirements. The user may select one or more optimization parameters and perform optimization using selected clinical phenotypic inputs and genetic inputs. Since the clinical trial requirements for various phases may be different, the user may select the phase of the clinical trial from box 266. One or more protocols for clinical trials may be provided in box 270. The user may select, for example, a plurality of pre-determined inclusion/exclusion criteria from box 274. The user may also specify a clinical trial design 278. For example, the user may select clinical trial designs such as single-blind trial, double-blind trial, crossover trial and open label trial. In a single-blinded trial, the participants do not know whether they're receiving a treatment or placebo (control) until the trial is over. In a double-blinded trial, neither the participants nor the researchers know who is receiving a treatment or a placebo until the trial is over. Sometimes, midway through the trial, the group receiving the treatment switches to the placebo, and vice versa, with neither group knowing which substance is which. This crossover is done to address ethical concerns about depriving one group of a possibly beneficial treatment for the duration of the trial. Crossover trial designs encourage trial participation by promising all participants access to the experimental treatment for half the trial's duration. In an open-label trial, everyone involved “sees the label” on the drug container and knows what he/she's taking.

The user may also have randomization options in box 286. The user may randomize the individuals to be involved in clinical trials. In one embodiment, the randomization may be performed within the selected individuals of similar genetic make-up. In another embodiment, the randomization may be performed within the selected individuals of similar clinical phenotypes.

According one embodiment of the invention, the system 44 may provide clinical trial recommendation based on optimization of clinical trial parameters utilizing clinical trial phenotypic input, genetic input and clinical trial requirements. In one embodiment, as illustrated in FIG. 5E, the user may run optimization and obtain recommendation of clinical trial by clicking box 290. According to another embodiment, the present invention may provide means for operating at least one phase of the clinical trial based on clinical trial recommendations.

While there are tools available to organizing information about commercial clinical trials—cost, billing, inclusion criteria, patients screened and entered into trials—, the present invention addresses the specific need for genetic information and provides for constructing, maintaining and monitoring clinical trials on this basis. This will have operational relevance to pharmaceutical, contract research organizations, site management organizations and clinical research specialists.

While a particular embodiment of the present invention has been described, it is to be understood that modifications will be apparent to those skilled in the art without departing from the spirit of the invention. The scope of the invention, therefore, is to be determined solely by the following claims.

The invention will be better understood by reference to the following non-limiting examples.

### EXAMPLE#1

A pharmaceutical company wishes to bring a lead compound targeted as an antidepressant into clinical trials. The system of the invention can be used to assist in such efforts.

In this example, the compound has already passed through Phase I trials and showed no limiting adverse events in normal controls. The pharmaceutical company desires to enter this compound into Phase II trials both to establish preliminary efficacy and dose finding. The pharmaceutical company may also wish to gain information regarding how the compound might successfully establish itself within a crowded but highly lucrative therapeutic area. Towards this end, the pharmaceutical company chooses to initiate a pharmacogenomic component to the Phase II trial in order to achieve preliminary indication that a specific genotype may predict favorable response to the drug. This genotype could be used to identify prospective patients in clinical settings who would benefit from the drug administration. In this way the pharmaceutical company establishes its initial market niche.

The pharmaceutical company may utilize the system of the present invention to design, operate and monitor the pharmacogenomic clinical trial. The company may utilize the system of the invention in the following fashion:

1. The compound may be known, for example, on the basis of its activity against in vitro targets to belong to a class of antidepressants which inhibit the activity of the serotonin transporter, the principle neuronal mechanism for terminating the physiological effect of the neurotransmitter serotonin once it is released into the synapse (space between two adjoining neurons). This group of agents is often referred to as selective serotonin reuptake inhibitors (SSRI's). In this way, this compound enhances and regulates the brain's serotonin system.

2. It is well known that drugs of this category, while effective, do not have similarly favorable effects on every patient. Indeed, some patients experience remarkably favorable effects, while other patients remain treatment resistant. Moreover, not all SSRI's have the same therapeutic profile in individual patients. Drug choice is largely related to empirical (trial and error) experience. Establishing a genetic marker which predicts favorable response to the antidepressant could establish a market segment for this compound.

3. The pharmaceutical company may utilize the CRT system 44 to access genotypic and phenotypic information on SSRI and examine what genetic variants are known to directly relate to the pathway (mechanism of action) of this compound.

4. The CRT system 44 informs the company that there are, for example, two common variants (long and short) in the promoter region of the serotonin transporter gene (5HTTLPR). The long variant has been reported to be associated with favorable response to an SSRI drug.

5. The system may also reveal, for example, several other gene variants, including but not limited to a functional variant in the tryptophan hydroxylase gene (TPH), and a functional variant in the promoter region of the dopamine transporter gene (DAT) which have relevance to brain function.

6. The pharmaceutical company may decide that the key gene target is the 5HTFLPR gene variant. The company has broad interest in the other gene variants as well.

7. The CRT system 44 may utilize its database to design a clinical study which balances frequencies of 5HTTLPR gene variants in study arms (placebo/active drug×2 doses). This may include the inclusion of patients who meet clinical criteria for the antidepressant trial and also include sufficient representation of patients with each of the two genotypes.

8. The CRT system 44 may allow for genotyping for the 5HTTLPR gene and selects from the resultant patient pools (which may include referrals received from contract research organizations and site management organizations) candidates for the study which are pre-genotyped for the target gene variant.

9. Patients chosen for the study on the basis of clinical and genotypic characteristics for the 5HTTLPR gene may also be genotyped for a selected group of exploratory gene variants (e.g., DAT).

10. The CRT system 44 may provide sufficient statistical power (distribution of 5HTTLPR long and short genotypes) regarding the potential use of this genotype as a predictor of drug response. The CRT system 44 may also enable a user in the pharmaceutical company to perform exploratory examination of additional gene variants.

11. The findings from this Phase II study may indicate a statistically or near statistically significant “signal” supporting the long variant of the 5HTTLPR as predictive of favorable drug response.

12. The pharmaceutical company may choose to advance clinical investigation for this compound into Phase III, using the now established dose, and may generate an a priori hypothesis regarding favorable drug response and the long form of the 5HTTLPR gene. This can be done in consultation with the Food and Drug Administration.

13. The pharmaceutical company may use the CRT system 44 to design and carry out the Phase III study in accordance with Food and Drug Administration (FDA) requirements. The validation and replication of the Phase III results suggests an application to the marketplace gain new patients who have a high probability of favorable response to the compound.

14. The CRT system 44 may enable the pharmaceutical company to include the results of the Phase III pharmacogenomic study in the company's New Drug Submission to the FDA.

15. Other data from exploratory gene variants in the Phase II study may suggest the value of some but not all gene variants. The company may decide to utilize this information as part of its preclinical drug discovery program.

### EXAMPLE#2

A pharmaceutical company may pursue a discovery program which focuses on the discovery of small molecules which can be used to improve cognitive function in patients with Mild Cognitive Impairment (MCI, a precursor to Alzheimer's disease) and to alter the course and severity of Alzheimer's Disease itself. The CRT system 44 of the invention can be used to assist in such discovery.

It has now been established with good medical confidence that a variant of the Apolipoprotein Gene, APOE E4 allele, results in dose-dependent (homozygosity>heterozygosity) increased risk for Alzheimer's disease including early age of onset and diminished response to currently available therapeutic agents. Nevertheless, this variant is not believed to reflect the core etiology for Alzheimer's disease and many patients develop both MCI and Alzheimer's disease who do not have this allele. For this reason, the pharmaceutical company may wish to bring new chemical entities into clinical trials. The pharmaceutical company may also wish to examine the relationship between gene variants which code for enzymes (e.g. beta secretase) and proteins which are intrinsically involved in the Alzheimer's pathological processes.

1. The pharmaceutical company may use the CRT system 44 to conceptualize and design a clinical trial for their new chemical entity which incorporates pharmacogenomic principles.

2. Because of the known effects of the APOE E4 allele the risk and course of MCI and Alzheimer's disease, the company may wish to carry out its Phase II trials using a clinical population of MCI patients and those with early Alzheimer's disease in which there is equal representation of patients with and without the APOE E4 allele. The company may utilize the CRT system 44 of the invention to create a pool of pre-genotyped patients who are then offered the opportunity to participate in the trial.

3. The pharmaceutical company may use the CRT system 44 to learn through the system's genotypic database 52 and retrieve additional information, for example, four other gene variants which have been reported to be related to the pathology of Alzheimer's disease.

4. As part of its exploratory Phase U study, the company may request patients to be pregenotyped for these four gene variants in addition to APOE E4 and decide to include four additional gene variants suggested by the company's scientists on the basis of pharmaceutical company's propriety discovery program.

5. The pharmaceutical company may utilize the CRT system 44 to provide information related to candidate genes, pharmacogenomic clinical trials design; it's selection of pre-genotyped patients is organized by the system. The CRT system 44 provides for the capacity to enter proprietary genetic information and to be used in the study.

6. In one scenario, the results of the phase II study may, for example, reveal a marked effect of APOB E4 status (negatively effecting treatment response) but also suggest, for example, two new gene variant predictors of favorable response.

7. Based upon these results, the pharmaceutical company may then design a Phase IIb study in which it intends to extend its early observations regarding new gene candidates, focusing on patients with MCI and Alzheimer's disease who do not have the APOE E4 allele. The CRT system 44 may further enable the pharmaceutical company to design the phase IIb study in accordance with FDA requirements.

8. The pharmaceutical company may then utilize the CRT system 44 to continue its development of this promising new chemical entity.

### EXAMPLE#3

A pharmaceutical company may be interested in pursuing treatment strategies for AIDS which will enhance current treatments aimed at delaying the onset of AIDS after an individual has positive immunoreactivity for the HW virus. The CRT system 44 of the invention can be used to assist in such efforts.

Despite the fact that AIDS in an infectious disease caused by a retrovirus, genetic host factors, similar to other infectious diseases, can greatly influence the clinical course of the disorder. Specifically, genetic variants of several chemokine receptors which effect the function of an individual's immune system appear to delay the onset of AIDS following exposure to the HW virus as reflected by positive immunoreactivity. For this reason, it is critical that the company control for known genetic causes of delayed AIDS onset in its treatment population in order to accurately determine its drug's therapeutic effects.

1. The pharmaceutical company may employ the CRT system 44 to determine known genetic variants known to alter the time course for the onset of AIDS.

2. The pharmaceutical company may wish to enter subjects with positive immunoreactivity to the HIV virus and be assured that genetic host factors are equally represented in all arms of the study.

3. The pharmaceutical company may employ the CRT system 44 to develop a pool of candidate patients who are also pre-genotyped for chemokine receptor gene variants. As patients continue to enter the study over the expected three years duration, the CRT system 44 may monitor the balance of genotypes in treatment arms.

4. The results of the study after, for example, three years may reveal that combination of “protective” genotypes and the experimental therapeutic agent result in <1% conversion to AIDS in comparison with a 25% conversion rate for patients without “protective” genotypes and who received the active experimental drug. Because of ethical considerations, placebo will not included in the study but placebo data, transposed from large public health AIDS databases, and may reveal >50% conversion rate for genetically cross-sectional analysis of positive immunreactive patients.

5. The pharmacogenomic approach which is enabled by the CRT system 44 may result in demonstration of synergistic effect of the experimental agent with host genetic factors and support the agent's superiority to the natural disease progression.

We claim:

1. A pharmacogenomic system for clinical trials, the system comprising:
a genotype database (GDB), the GDB comprising genetic information for a plurality of patients;
a clinical database (CDB), the CDB comprising clinical phenotypic information for a plurality of patients;
a clinical trial requirement database (CRDB), the CRDB comprising information on clinical trial requirements for at least one phase of a clinical trial;
association modules that are connected to GDB, and CDB and are adopted to determine an association between the genetic information and the clinical phenotypic information for a plurality of patients; and
recommendation modules that are connected to GDB, CDB, and CRDB and adopted to provide clinical trial recommendations utilizing the genetic information, the clinical phenotypic information, the clinical trial requirement information and the determined association between the clinical information and the genetic information.

2. The system of claim 1 further comprising selection modules that are connected to the system for selecting one or more patients based on the genetic information, wherein the selection is performed using plurality of statistical methods.

3. The system of claim 1, wherein the genetic information correspond to one or more variation in candidate genes.

4. The system of claim 1, wherein the genetic information correspond to plurality of Single Nucleotide Polymorphisms.

5. The system of claim 1, wherein the clinical trial requirement information correspond to one or more protocols for Phase I of a clinical trial.

6. The system of claim 1, wherein the clinical trial requirement information correspond to one or more inclusion/exclusion criteria for clinical trials.

7. The system of claim 1, wherein the association is determined my one or more of pre-determined statistical methods.

8. The system of claim 1, wherein the recommendation modules perform optimization of clinical trial parameters for providing clinical trial recommendations.

9. A pharmacogenomic system for clinical trials, the system comprising:
a genotype database (GDB), the GDB comprising genetic information for a plurality of patients;
a clinical database (CDB), the CDB comprising clinical phenotypic information for a plurality of patients;
a clinical trial requirement database (CRDB), the CRDB comprising information on clinical trial requirements for at least one phase of a clinical trial;
association means that are connected to GDB, and CDB and are adopted to determine an association between the genetic information and the clinical phenotypic information for a plurality of patients; and
recommendation means that are connected to GDB, CDB, and CRDB and adopted to provide clinical trial recommendations utilizing the genetic information, the clinical phenotypic information, the clinical trial requirement information and the determined association between the clinical information and the genetic information.

10. The system of claim 9 further comprising selection means for selecting one or more patients based on the genetic information, wherein the selection is performed using plurality of statistical methods.

11. The system of claim 9, wherein the genetic information correspond to one or more variation in candidate genes.

12. The system of claim 9, wherein the genetic information correspond to plurality of Single Nucleotide Polymorphisms.

13. The system of claim 9, wherein the clinical trial requirement information correspond to one or more protocols for Phase I of a clinical trial.

14. The system of claim 9, wherein the clinical trial requirement information correspond to one or more inclusion/exclusion criteria for clinical trials.

15. The system of claim 9, wherein the association is determined my one or more of pre-determined statistical methods.

16. The system of claim 9, wherein the recommendation means perform optimization of clinical trial parameters for providing clinical trial recommendations.

17. A pharmacogenomic method for clinical trials, the method comprising the steps of:
enabling a user to access a genotype database (GDB), the GDB comprising genetic information for a plurality of patients;
enabling a user to access a clinical database (CDB), the CDB comprising clinical phenotypic information for a plurality of patients;
enabling a user to access a clinical trial requirement database (CRDB), the CRDB comprising information on clinical trial requirements for at least one phase of a clinical trial;
enabling a user to determine an association between the genetic information and the clinical phenotypic information for a plurality of patients; and
enabling a user to cause the system to provide clinical trial recommendations utilizing the genetic information, the clinical phenotypic information, the clinical trial requirement information and the determined association between the clinical information and the genetic information.

18. The method of claim 17 further comprising a step of enabling a user to select one or more patients based on the genetic information, wherein the selection is performed using plurality of statistical methods.

19. The method of claim 17, wherein the genetic information correspond to one or more variation in candidate genes.

20. The method of claim 17, wherein the genetic information correspond to plurality of Single Nucleotide Polymorphisms.

21. The method of claim 17, wherein the clinical trial requirement information correspond to one or more protocols for Phase I of a clinical trial.

22. The method of claim 17, wherein the clinical trial requirement information correspond to one or more inclusion/exclusion criteria for clinical trials.

23. The method of claim 17, wherein the association is determined my one or more of predetermined statistical methods.

24. The method of claim 17, wherein method performs optimization of clinical trial parameters for providing clinical trial recommendations.

25. A processor readable pharmacogenomic medium for clinical trials, said processor readable medium comprising:
a first processor readable program code for enabling a user to access a genotype database (GDB), the GDB comprising genetic information for a plurality of patients;
a second processor readable program code for enabling a user to access a clinical database (CDB), the CDB comprising clinical phenotypic information for a plurality of patients;
a third processor readable program code for enabling a user to access a clinical trial requirement database (CRDB), the CRDB comprising information on clinical trial requirements for at least one phase of a clinical trial;
a fourth processor readable program code for enabling a user to determine an association between the genetic information and the clinical phenotypic information for a plurality of patients; and
a fifth processor readable program code for enabling a user to cause the system to provide clinical trial recommendations utilizing the genetic information, the clinical phenotypic information, the clinical trial requirement information and the determined association between the clinical information and the genetic information.

26. A pharmacogenomic system for clinical trials, the system comprising:
means for providing genetic information for a plurality of patients;
means for providing clinical phenotypic information for a plurality of patients;
means for providing information on clinical trial requirements for at least one phase of a clinical trial;
association modules that determine an association between the genetic information and the clinical phenotypic information for a plurality of patients; and
recommendation modules that provide clinical trial recommendations utilizing the genetic information, the clinical phenotypic information, the clinical trial requirement information and the determined association between the clinical information and the genetic information.
